(19) United States
(12) Patent Application Publication
(10) Pub. No.: US 2025/0135042 A1
(43) Pub. Date: May 1, 2025

(54) Perfluorocarbon-Containing Contrast Agent for Use in MRI Imaging of Body Cavities

(71) Applicant: University of Pittsburgh - Of the Commonwealth System of Higher Education, Pittsburgh, PA (US)

(72) Inventors: Pradeep Tyagi, Pittsburgh, PA (US); Chan-Hong Moon, Pittsburgh, PA (US)

(21) Appl. No.: 18/925,158

(22) Filed: Oct. 24, 2024

Related U.S. Application Data
(60) Provisional application No. 63/593,629, filed on Oct. 27, 2023.

(51) Int. Cl. A61K 49/10

(52) U.S. Cl. CPC A61K 49/108; A61K 49/10; A61K 49/1806

(57) ABSTRACT

Provided herein is a contrast agent composition comprising a contrast agent, such as a gadolinium chelate contrast agent, and a perfluorocarbon. Provided herein also is a method of imaging a patient's bladder or other organs containing a cavity, using MRI using a contrast agent composition comprising a contrast agent, such as a gadolinium chelate contrast agent, and a perfluorocarbon.

### CROSS REFERENCE TO RELATED APPLICATIONS

This application claims priority to U.S. Provisional Patent Application No. 63/593,629 filed Oct. 27, 2023, which is incorporated herein by reference in its entirety.

### STATEMENT REGARDING FEDERAL FUNDING

This invention was made with government support under Grant No. CA263243 awarded by the National Institutes of Health. The government has certain rights in the invention.

Diseased lesions of the bladder wall, a thin-walled soft tissue, are challenging to image using available imaging modalities. Superior soft tissue resolution of MRI presents an opportunity that can only be realized with an innovation that increases the signal of bladder wall while simultaneously decreasing or masking signals in the bladder lumen, providing improved image contrast with readily-discernable lesions. While signal in contrast-enhanced MRI is dependent on the tissue concentration of contrast agent delivered to tissue by perfusion after intravenous injection, it is challenging to control the dynamic concentration of contrast agent at the disease site of target region after injection. However, administration of contrast mixture via an intravesical route or localized administration into body cavities offer better management for selective brightening of the lesioned bladder wall in T1 weighted images.

Intravenously injected gadolinium chelate can successfully improve image resolution for certain organs other than the bladder, but there is a complication in injecting gadolinium chelate for bladder wall imaging because injected chelates arrive transiently at the bladder wall immediately after injection via perfusion, and then the arrival of excreted gadolinium chelate in urine confounds the image contrast of lesion. Since contrast enhancement in MRI is a function of gadolinium chelate (e.g., Gadobutrol) concentration in the tissue of interest and the biological nuances of the bladder lining and of other accessible body cavities allow safe escalation of concentrations that are >10 fold higher than plasma concentration of gadolinium chelate achieved routinely via the systemic route, e.g., intravenous injection, there are intrinsic advantages with localized administration of gadolinium chelates which can only be realized in combination with another agent capable of causing the signal decay in lumen without artifacts.

Superior contrast agent formulations are therefore needed for imaging body cavities, e.g. organs with cavities, such as the bladder.

### SUMMARY

According to one aspect or embodiment of the present invention, a contrast agent composition, e.g., an emulsion, is provided, comprising at least 10 mM (millimolar) of a contrast agent, such as a gadolinium chelate contrast agent, in an emulsion comprising at least 10% w/v of a perfluorocarbon.

According to another aspect or embodiment of the present invention, a method of imaging an organ having a cavity of a patient, comprising administering an emulsion composition comprising at least 10 mM (millimolar) of a contrast agent, such as a gadolinium chelate contrast agent, in an emulsion comprising at least 10% w/v of a perfluorocarbon into a cavity of the patient's organ having a cavity, and obtaining a magnetic resonance imaging image of the patient's organ having a cavity containing the emulsion composition.

According to another aspect or embodiment of the present invention, an MRI method comprising imaging an organ having a cavity of a patient, comprising administering an emulsion composition comprising at least 10 mM (millimolar) of a contrast agent, such as a gadolinium chelate contrast agent, in an emulsion comprising at least 10% w/v of a perfluorocarbon into a cavity of the patient's organ having a cavity, and obtaining a magnetic resonance imaging image of the patient's organ having a cavity containing the emulsion composition.

In another aspect or embodiment of the present invention, a method of imaging a bladder of a patient is provided, comprising administering an emulsion composition comprising at least 10% w/v of a perfluorocarbon into the patient's bladder, and obtaining a magnetic resonance imaging image of the patient's bladder containing the emulsion composition.

The following numbered clauses outline various aspects or embodiments of the present invention.

Clause 1. A contrast agent emulsion composition comprising at least 10 mM (millimolar) of a contrast agent in an emulsion comprising at least 10% w/v of a perfluorocarbon.

Clause 2. The emulsion composition of clause 1, wherein the contrast agent comprises a gadolinium chelate contrast agent.

Clause 3. The emulsion composition of clause 2, wherein the contrast agent comprises gadopentetate dimeglumine, gadobenate dimeglumine, gadoxetate disodium, gadofosveset trisodium, gadodiamide, gadoversetamide, gadoterate meglumine, gadoteridol, gadobutrol, or gadopiclenol.

Clause 4. The emulsion composition of clause 1, in the form of an emulsion comprising in a final volume: from 5 to 20 mg/ml of a gadolinium chelate, from 10 to 20% w/v of a perfluorocarbon, from 3.5% to 6% w/v of an emulsifier, surfactant, and/or one or more other lipids, and an aqueous phase to the final volume.

Clause 5. The emulsion composition of clause 4, wherein the emulsifier, surfactant, and/or one or more other lipids are selected from lecithin, a poloxamer nonionic detergent, and amphiphilic lipid composition comprising a monoglyceride, a diglyceride, and/or a triglyceride modified with a hydrophilic moiety, such as a polyoxyethylene (PEG)-containing moiety, e.g., a transesterified ethoxylated vegetable oil.

Clause 6. The emulsion composition of clause 5, comprising: a nonionic surfactant such as a poloxamer nonionic surfactant (a polyoxyethylene-polyoxypropylene block copolymer, often referred to as a pluronic surfactant), and one or both of capryl caproyl polyoxyl-8 glycerides and oleoyl polyoxyl-6 glycerides.

Clause 7. The emulsion composition of clause 1, comprising: from 5 to 20 mg/mL of the gadolinium chelate; from 10 to 20% w/v of the perfluorocarbon; from 1 to 2% w/v of lecithin; from 1 to 2% w/v of the poloxamer nonionic surfactant, from 1.5 to 2% w/v of capryl caproyl polyoxyl-8 glycerides and/or oleoyl polyoxyl-6 glycerides; and an aqueous phase, to the final volume.

Clause 8. The emulsion composition of any one of clauses 1-7, comprising perfluorodecalin.

Clause 9. The emulsion composition of clause 8, comprising ≥10% w/v perfluorodecalin.

Clause 10. The emulsion composition of any one of clauses 1-9, comprising perfluorodecalin, lecithin, a poloxamer, and a transesterified ethoxylated oil.

Clause 11. A method of imaging an organ having a cavity of a patient, comprising administering an emulsion composition according to any one of claims 1-10 into a cavity of the patient's organ having a cavity, and obtaining a magnetic resonance imaging image of the patient's organ having a cavity containing the emulsion composition.

Clause 12. The method of clause 11, wherein the organ having a cavity is a lumen of a patient's bladder.

Clause 13. The method of clause 12, wherein the emulsion composition is administered transurethrally to the patient's bladder.

Clause 14. The method of clause 11, wherein the organ having a cavity is the patient's GI tract, the patient's respiratory tract, the patient's heart, a blood vessel of the patient, or a bursa of the patient.

Clause 15. The method of any one of clauses 11-14, wherein the MRI image is obtained using an MRI scanner of at least 1.5 T (1.5 Tesla), or at least 3 T.

Clause 16. The method of clause 15, wherein the MRI scanner is a 7 T MRI scanner.

Clause 17. The method of any one of clauses 11-16, wherein the contrast agent comprises a gadolinium chelate contrast agent.

Clause 18. The method of clause 17, wherein the contrast agent comprises one or more of gadopentetate dimeglumine, gadobenate dimeglumine, gadoxetate disodium, gadofosveset trisodium, gadodiamide, gadoversetamide, gadoterate meglumine, gadoteridol, gadobutrol, or gadopiclenol.

Clause 19. The method of any one of clauses 11-18, wherein the emulsion composition comprises in a final volume: from 5 to 20 mg/ml of a gadolinium chelate, from 10 to 20% w/v of a perfluorocarbon, from 3.5% to 6% w/v of an emulsifier, surfactant, and/or one or more other lipids, and an aqueous phase to the final volume.

Clause 20. The method of clause 19, wherein the emulsifier, surfactant, and/or one or more other lipids are selected from lecithin, a poloxamer nonionic detergent, and amphiphilic lipid composition comprising a monoglyceride, a diglyceride, and/or a triglyceride modified with a hydrophilic moiety, such as a polyoxyethylene (PEG)-containing moiety, e.g., a transesterified ethoxylated vegetable oil.

Clause 21. The method of clause 20, wherein the emulsion composition comprises: a nonionic surfactant such as a poloxamer nonionic surfactant, and one or both of capryl caproyl polyoxyl-8 glycerides and oleoyl polyoxyl-6 glycerides.

Clause 22. The method of clause 19, wherein the emulsion composition comprises: from 5 to 20 mg/mL of the gadolinium chelate; from 10 to 20% w/v of the perfluorocarbon; from 1 to 2% w/v of lecithin; from 1 to 2% w/v of the poloxamer nonionic surfactant, from 1.5 to 2% w/v of capryl caproyl polyoxyl-8 glycerides and/or oleoyl polyoxyl-6 glycerides; and an aqueous phase, to the final volume.

Clause 23. The method of any one of clauses 11-22, wherein the emulsion composition comprises perfluorodecalin.

Clause 24. The method of clause 23, the composition comprising ≥10% w/v perfluorodecalin.

Clause 25. A method of imaging a bladder of a patient, comprising: administering an emulsion composition comprising at least 10% w/v of a perfluorocarbon into the patient's bladder, and obtaining a magnetic resonance imaging image of the patient's bladder containing the emulsion composition.

### BRIEF DESCRIPTION OF THE DRAWINGS

FIG. 1 provides images of gadobutrol infusion of bladder phantom constructed of 12% polyacrylamide gel. While both Ferumoxytol and PFC can cause signal decay within cavity appearing as bladder (phantom) without suppressing the signal of gadobutrol that diffuses away in T2 weighted MRI, only PFC managed to cause signal decay within cavity (bladder phantom) in both T2 weighted (spin echo) and T1 weighted (gradient echo) image acquisition for MRI. Ferumoxytol could not darken bladder phantom in T1 weighted MRI.

FIGS. 2A and 2B: Graphical plots and T1 weighted images of individual tubes containing serial two-fold dilutions of Gadobutrol without ≥10% w/v PFC emulsion (FIG. 2A) or with ≥10% w/v PFC emulsion (FIG. 2B) display that ≥10% PFC emulsion diminishes the ¹H relaxivity of Gadobutrol from 3.95 L/mmol/s (FIG. 2A) to 2.05 L/mmol/s (FIG. 2B) at 7 Tesla scanner. T1 relaxation rate of water protons (R₁=1/T₁) was calculated from the slope of the line plotted by computing T1 relaxation time from T1 weighted images acquired at identical magnetic field strength of 7 Tesla at 37° C.

FIGS. 3A and 3B: in vivo application of PFC mediated signal decay in lumen. FIG. 3A—Spin echo images acquired with identical pulse sequence at 7 T before (FIG. 3A (A)) and after (FIG. 3A (B)) transurethral instillation of 0.05 mL of ≥10% w/v PFC emulsion mixed with Gadobutrol 20 mM or just ≥10% w/v PFC emulsion (FIG. 3A (C)). Grey bar panel in each figure displays signal intensity and ≥10% w/v PFC emulsion (FIG. 3A (C)) enhances the signal intensity in bladder lumen relative to FIG. 3A (A) but the entrapment of 20 mM Gadobutrol with ≥10% w/v PFC emulsion (FIG. 3A (B)) generated unexpected results by decaying the signal within lumen but brightening the lesion of bladder cancer on the bladder wall. FIG. 3B provides multi slice imaging of the tumor in presence of urine in lumen FIG. 3B (A) and in presence of contrast mixture instilled into lumen FIG. 3B (B) by insertion of catheter into urethra in slice seen in FIG. 3A. Slices shown in FIGS. 3A and 3B are taken 1.6 mm apart.

FIG. 4: T1 weighted (panel A) and T2 weighted (panel B) image acquisition of bladder phantom filled with 5 mL of ≥10% w/v PFC emulsion alone (left most image) or mixed with ascending concentrations of Gadobutrol 2 mM from left to right. T1 weighted and T2 weighted signal intensity of the filled space enclosed by blue and orange ellipse, respectively is plotted by the curves of respective color in Panel C displays that while ≥10% PFC emulsion attenuates T2 weighted signal intensity marked by complete loss of signal beyond 5 mM there is a simultaneous accentuation of T1 weighted signal intensity till plateau is reached beyond 10 mM.

FIGS. 5A and 5B: Advantage of PFC over Ferumoxytol: FIG. 5A—Irrespective of the field strength, Ferumoxytol induces MRI artifacts in the image field outside of bladder wall with spin echo pulse sequence for T2-weighted MRI at 7 Tesla (Panel B) and T1-weighted image acquisition at 3 Tesla (Panels C-D, FIG. 5B). Comparative spin echo images of a rodent bladder instilled with Gadobutrol 20 mM mixed with ≥10% PFC emulsion (Panel A) or 5 mM Ferumoxytol (Panel B) at 7 T display that ≥10% w/v PFC emulsion generates positive contrast of the bladder wall without inducing the multiple local susceptibility-induced distortions of magnetic field visible as alternating regions of darkness and brightness without any tangible basis such as pathology or the presence of contrast agent outside the bladder wall (Panels B-D).

### DETAILED DESCRIPTION

Other than in the operating examples, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements. Furthermore, when numerical ranges of varying scope are set forth herein, it is contemplated that any combination of these values inclusive of the recited values may be used.

Also, it should be understood that any numerical range recited herein is intended to include all sub-ranges subsumed therein. For example, a range of “1 to 10” is intended to include all sub-ranges between and including the recited minimum value of 1 and the recited maximum value of 10, that is, having a minimum value equal to or greater than 1 and a maximum value of equal to or less than 10.

As used herein “a” and “an” refer to one or more.

As used herein, the term “comprising” is open-ended and may be synonymous with “including”, “containing”, or “characterized by”.

As used herein, the term “polymer composition” is a composition comprising one or more polymers. As a class, “polymers” includes, without limitation, homopolymers, heteropolymers, co-polymers, block polymers, block co-polymers and can be both natural and synthetic. Homopolymers contain one type of building block, or monomer, whereas co-polymers contain more than one type of monomer.

A polymer “comprises” or is “derived from” a stated monomer if that monomer is incorporated into the polymer. Thus, the incorporated monomer that the polymer comprises is not the same as the monomer prior to incorporation into the polymer, in that at the very least, during incorporation of the monomer, certain groups, e.g., terminal groups, that are modified during polymerization are changed, removed, and/or relocated, and certain bonds may be added, removed, and/or modified. An incorporated monomer is referred to as a “residue” of that monomer. A polymer is said to comprise a specific type of linkage if that linkage is present in the polymer. Unless indicated otherwise, Mw refers to the weight average molecular weight of a polymer composition, or where otherwise applicable where simple molecular mass calculated from the atomic weights of the constituent atoms of a molecule. A “moiety” is a portion of a compound, and includes a residue or group of residues within a larger polymer.

By “biocompatible”, in the context of the compositions described herein, it is meant that a composition and degradation products thereof, is essentially, practically (for its intended use) and/or substantially non-toxic, non-injurious or non-inhibiting or non-inhibitory to cells, tissues, organs, and/or organ systems that would come into contact with the composition. In the context of the compositions described herein, the compositions and their constituents are biocompatible, non-toxic, and/or pharmaceutically-acceptable in that they are safe for use in humans and/or animals—meaning that they are non-toxic, or substantially non-toxic within acceptable tolerances within the medical, pharmaceutical, and/or veterinary fields, such as according to requirements of governmental regulatory agencies for medical, pharmaceutical, and/or veterinary uses, such as the United States Food and Drug Administration.

A “drug substance”, “therapeutic agent” or “active agent” means an active ingredient that is intended to furnish pharmacological activity or other direct effect in the diagnosis, cure, mitigation, treatment, or prevention of disease or to affect the structure or any function of the human body or animal. A “drug product” is a finished dosage form, for example, tablet, capsule, or solution that contains a drug substance, generally, but not necessarily, in association with one or more other ingredients. A drug product may be a composition of matter, object, or device, such as a liquid, solid, powder, capsule, tablet, ointment, cream, injectable, aerosol, patch, or any other physical form that is used to deliver an active agent, and can include single or multiple doses. Thus, in the example of a gadolinium-containing contrast agent, including a perfluorocarbon, the drug substance or active agent is the gadolinium-containing contrast agent, while the drug product is the composition, or a single-use dispenser containing the ingredients for preparation of a composition comprising the gadolinium-containing contrast agent and a perfluorocarbon, and an emulsifier. A “dosage form” refers to the form in which the drug product is marketed for use, e.g. as sealed ampule, catheter and reservoir, a capsule, tablet, inhaler, nebulizer, drops, etc., and the drug product may be provided in two phases to be sonicated immediately prior to use—that is, in sufficient time prior to administration such that the ingredients and emulsion remain stable until delivery to a patient.

Provided herein is a novel composition to advance the treatment of urinary bladder cancer by enabling better staging of the cancer compared to present techniques by combining the “positive” MRI contrast effects of a standard gadolinium chelate contrast agent and the “negative” contrast stemming from diamagnetic characteristics of perfluorocarbons formulated in the emulsion. The emulsion mixed with gadolinium chelate contrast agent is instilled in the bladder instead of the traditional approach of injecting into the vein. Therefore, the imaging approach overcomes the dependence on bladder perfusion for contrast enhancement of malignant or inflamed lesions as gadolinium contrast agent is delivered by diffusion to disease site in the bladder wall. Accordingly, our technique can be used on patients with a sensitive bladder who cannot tolerate bladder distension to 300 mL for brightening lesion after intravenous injected gadolinium chelate in MRI. Since perfluorocarbon emulsion has been FDA approved for systemic use in the past and we envision local instillation in bladder, with minimal systemic toxicity concerns. Imaging approach can also be developed as a bladder permeability assay for benign lesions and for staging malignant lesions in clinical decision making.

There is no commercial bladder permeability assay available for aiding clinical diagnosis of disease and treatment decisions. The approach provided herein provides a viable path to reach that goal based on animal studies. The described imaging approach overcomes the dependence over perfusion for imaging bladder lesions and can brighten poorly perfused lesion through diffusion driven by a concentration gradient of gadolinium chelate from the bladder lumen. Since diffused gadolinium chelate is cleared by veins and not by arteries, this imaging approach allows imaging of venous stasis and radiation-free angiography of the bladder. The difference in the rate of gadolinium chelate diffusion into normal and diseased area can be a robust indicator of permeability and perfusion of normal and diseased regions of interest. The formulation comprises a perfluorocarbon emulsion that overcomes the MRI artifacts associated with the use of Ferumoxytol. This imaging approach can be used for surgical free cancer staging of indolent bladder cancer in elderly patients unlikely to tolerate anesthesia or survive surgery.

As used herein the “bladder lumen” refers to the inside space within the bladder which acts as a reservoir for urine. An “organ having a cavity” is any organ having a cavity, with bladder (e.g., bladder lumen) being an example. Additional organs having a cavity include the GI tract, vagina, urethra, the respiratory tract, the heart, a blood vessel, or bursae.

A gadolinium contrast agent is any contrast agent comprising a gadolinium chelate useful for magnetic resonance imaging methods, and having an overall molecular weight of 1000 Da or less, and/or a Stokes-Einstein radius of 1 nm (nanometer(s)) or less or 0.5 nm or less, including any increment thereof, such as 0.4 nm. The gadolinium chelates may be linear or macrocyclic, ionic, or non-ionic, e.g., linear-ionic, linear-nonionic, macrocyclic-ionic, or macrocyclic-nonionic. Specific examples of gadolinium contrast agents include, without limitation: Gd-DTPA, gadopentetate dimeglumine (Magnevist); Gd-BOPTA, gadobenate dimeglumine (MultiHance); Gd-EOB-DTPA, gadoxetate disodium (Eovist, Primovist); MS325, gadofosveset trisodium (Vasovist, Ablavar); Gd-DTPA-BMA, gadodiamide (Omniscan); Gd-DTPA-BMEA, gadoversetamide (OptiMARK); Gd-DOTA, gadoterate meglumine (Dotarem, Artirem); Gd-HP-DO3A, gadoteridol (ProHance); Gd-BT-DO3A, gadobutrol (Gadovist, Gadavist); or gadopiclenol (Elucirem, Vueway).

“Perfluorocarbons” are clear, odorless liquids composed primarily of covalently bonded carbon and fluorine atoms, having, for example and without limitation, the formula CₓF_{y}, including ethers thereof. A perfluorocarbon having one or more ether linkages is termed a “perfluoroether”, and can be cyclic (perfluorocycloether). Perfluorocarbons may include single compounds and mixtures of compounds, such as a mixture of perfluorocycloether and perfluorooctane. Perfluorocarbons are broadly-known, and many are commercially available, including those that have been studied for medical or pharmacological purposes, including breathing, respiratory therapies (e.g. liquid ventilation, or LV), imaging, and drug delivery (see, e.g., Holman R, et al. Perfluorocarbon Emulsion Contrast Agents: A Mini Review. Front Chem. 2022 Jan. 10; 9:810029). Perfluorocarbons as a class include substituted perfluorocarbons in which one or more heteroatoms are added, such as, for example and without limitation, 0 in the case of ethers, and Br such as in the case of Perflubron (perfluorooctylbromide). Perfluorocarbons may be linear, branched, cyclic, or combinations thereof and are saturated, and in various aspects include from six to twelve carbon atoms. Although there are many different types of PFCs suitable for delivery to the bladder as described herein, many clinical LV trials have utilized Perflubron (perfluorooctyl bromide). In one example, the PFC may be perfluorodecalin, a hydrophobic, viscous oil with high specific gravity ranging from 1.76 to 2.03 and low surface tension which merited FDA approval as an intraoperative tool in vitreoretinal surgery (see, e.g., Choi H, Ma J. Use of perfluorocarbon compound in the endorectal coil to improve MR spectroscopy of the prostate. AJR Am J Roentgenol. 2008 April; 190(4):1055-9; PMID: 18356455). LV work done in animals has also utilized FC-77 (mixture of perfluorocycloether and perfluorooctane). The perfluorocarbon may be perfluoro-15-crown-5 ether or PFPE oxide (see, e.g., U.S. Pat. No. 9,352,057, incorporated herein by reference for its technical disclosure). The perfluorocarbons described herein, as well as other compositions useful in forming the drug-delivery emulsion composition described herein, such as an emulsifier, and the constituents of the aqueous phase, may be biocompatible, non-toxic, and/or pharmaceutically-acceptable.

Due primarily to the extremely strong covalent bonding between carbon and fluorine atoms (C—F bonds are 485 kJ/mol), PFCs have high intramolecular forces and tend to be very stable. In a biological environment, this translates to PFCs being bioinert and resisting any type of metabolism or enzymatic changes. PFCs are also nonpolar and tend to have very low intermolecular, or van der Waals, forces. These low intermolecular forces are responsible for many of the properties of PFC. First, due to these weak forces, PFCs tend to be quite volatile. Second, weak van der Waals forces also give PFCs a very low surface tension (<20 dyne/cm) which make PFC an excellent solvent for gases such as oxygen and carbon dioxide (solubility of ˜50 mL 02 and 140-210 mL CO₂ per dL of PFC). PFCs are also fairly dense (about twice that of water) and not soluble with water or lipids. As such, PFCs are immiscible with virtually all physiological substances other than gases.

Although small amounts of PFC may be transported across the organ wall into systemic circulation, this amount is typically less than 1% of the administered dose. Virtually all delivered PFC is believed to ultimately leave the body via evaporation through the lung or transpiration through the skin. Even PFC delivered to the systemic circulation in the form of an emulsion for use as an imaging agent or blood substitute has been shown to be cleared via expired air after phagocytosis by reticuloendothelial macrophages. Although trace amounts of PFC have been shown to preferentially accumulate in fatty tissues and remain for relatively long periods of time, there has been no evidence of any negative consequences. Along the same lines, the long-term effects and toxicity of PFC have been studied extensively in animals and patients for periods up to 10 years without evidence of adverse effects. Further description of perfluorocarbons and their benefits are described in Orizondo, R. A. (2015) Antibacterial Perfluorocarbon Ventilation: A Novel Treatment Method for Bacterial Respiratory Infections (Doctoral dissertation). Retrieved from hdl.handle.net/2027.42/116728 and in U.S. Pat. No. 10,322,184.

An emulsifier is a compound or composition that facilitates production of a stable emulsion of a perfluorocarbon and an aqueous phase, such as water, saline (e.g. normal saline), phosphate-buffered saline (PBS), a salt solution, optionally comprising a water-soluble diagnostic agent or composition, such as a gadolinium contrast agent as described herein. The emulsifier may be any useful emulsifier, surfactant, or combination thereof effective to produce an emulsion, e.g., a stable emulsion. Emulsifiers may be amphiphilic, e.g., including both hydrophilic and hydrophobic portions. The emulsion may comprise soya lecithin (e.g., lipoid egg phosphatidyl choline), cholesterol, or dipalmitoyl phosphatidylethanolamine (DPPE) or 1,2-dipalmitoyl-sn-glycero-3-phospho-(1′-myo-inositol) (DPPI). For example and without limitation, the emulsion may comprise a surfactant mixture (mixture of surfactant(s) and/or emulsifier(s)) comprising Pluronic F-68, soya lecithin, capryl caproyl polyoxyl-8 glycerides, oleoyl polyoxyl-6 glycerides, or 70 mol % of lecithin; 28 mol % of cholesterol; and 2 mol % of DPPI or DPPE. Additional additives, such as propylene glycol may be included in the emulsion formulation. The emulsifier may be any useful emulsifier, surfactant, or combination thereof effective to produce an emulsion, e.g., a stable emulsion.

An “emulsion” is a stable mixture of two or more immiscible liquids held in suspension by an emulsifier. The emulsion comprises a continuous phase and a dispersed phase dispersed, e.g., as droplets, within the continuous phase. In the context of the present invention, the two immiscible liquids may be the perfluorocarbon and the aqueous phase, for example and without limitation, comprising a gadolinium contrast agent in any suitable form, and an emulsifier as described herein. For example, the aqueous phase may form the continuous phase and the perfluorocarbon may be dispersed within the aqueous phase. Emulsions can be formed by a variety of processes as are known in the art, for example and without limitation by physical mixing, e.g., shaking, stirring, blending, or homogenizing, or by sonication, e.g., by ultrasonication. Pharmaceutically-acceptable emulsions are often produced by sonicating a two-phase composition prior to use, e.g., at the bedside or pharmacy, such as in a hospital pharmacy immediately prior to use. A “stable emulsion” in the context of the present invention is an emulsion that substantially remains in an emulsified state prior to delivery of the emulsion into the bladder or luminal organ of a patient, and therefore remains an emulsion at, for example, from 20° C. to 37° C. for a time period sufficiently long enough for MRI imaging of the luminal organ, e.g., a bladder of a patient. Stability of an emulsion may be measured by obtaining a mid-level sample of an emulsified solution allowed to sit in a centrifuge tube without agitation at room temperature for five minutes after emulsification, and determining if an effective amount of the gadolinium contrast agent remains in that mid-level sample. If the solution does not resolve into two phases, and a therapeutically-effective (e.g., diagnostically-effective) amount of the contrast agent remains in the sample, then the emulsion is considered stable.

Emulsions may be formed in any useful manner, as is broadly-known in the pharmacology field (see, e.g., Remington: The Science and Practice of Pharmacy, 21^{st} edition, ed. Paul Beringer et al., Lippincott, Williams & Wilkins, Baltimore, MD Easton, Pa. (2005), Chapter 39). In addition to the PFC described herein, an emulsion may comprise an emulsifier composition, a surfactant composition, and/or a lipid composition. Though categorized separately here for convenience, it is understood that emulsifiers, surfactants, and lipids have significantly overlapping definitions and properties that may result in individual components fitting multiple definitions. The characterization of any components here within a category should not be seen to limit their use in any other category in which they could be reasonably used, and components should be seen as interchangeable based on what is known in the art. In one non-limiting example, an emulsion may comprise lecithin, cholesterol, or dipalmitoyl phosphatidylethanolamine (DPPE) or other amphiphilic compounds or compositions, such as 1,2-dipalmitoyl-sn-glycero-3-phospho-(1′-myo-inositol) (DPPI). For example, and without limitation, an emulsion composition may comprise a mixture of surfactant(s) and/or emulsifier(s)) comprising Pluronic F-68, soya lecithin, capryl caproyl polyoxyl-8 glycerides, oleoyl polyoxyl-6 glycerides, or 70 mol % of lecithin; 28 mol % of cholesterol; and 2 mol % of DPPI or DPPE. Additional additives, such as propylene glycol, capryl caproyl polyoxyl-8 glycerides, and/or oleoyl polyoxyl-6 glycerides may be included in the emulsion formulation.

A perfluorocarbon emulsion comprises a perfluorocarbon compound, and one or more additional emulsifier compounds or compositions. For example, an emulsion may comprise one or more of: Pluronic F-68, soya lecithin, and a transesterified ethoxylated vegetable oil such as capryl caproyl polyoxyl-8 glycerides (e.g., a LABRASOL©, Gattefosse), and/or oleoyl polyoxyl-6 glycerides (e.g., a LABRAFIL©, Gattefosse)), an aqueous solution, such as purified water, and, optionally, a gadolinium contrast agent, each in amounts effective to produce a stable emulsion for use as a bladder contrast composition. In exemplary formulations described herein, the emulsion may comprise at least 10 mM (millimolar), at least 15 mM, or about 20 mM gadolinium chelate, e.g., Gadobutrol. The formulation also comprises 10% or more (10%) weight volume (w/v) of a perfluorocarbon compound.

A surfactant, or a surface-active agent, is a compound which reduces the interfacial tension between two substances, facilitating the mixing of the substances. Surfactants are often amphiphilic, containing both hydrophilic and hydrophobic portions. In emulsions, surfactants may also serve as emulsifiers or as part of an emulsifier composition. An emulsifier may include a fluorosurfactant which may include a fluorophilic segment, such as a perfluorocarbon segment, block, or moiety, that partitions to a perfluorocarbon, and a hydrophilic segment, block, or moiety, such as a linear or branched block copolymer of a perfluorocarbon and a poly(C₂-C₄ alkyl)ether, such as a polyoxyethylene, polyoxyproplyene, or polyoxybutylene. Copolymer block size, polydispersity, and overall Mw of the block copolymer can be optimized for any emulsion composition. As described in Fabiilli M L, et al. (Delivery of water-soluble drugs using acoustically triggered perfluorocarbon double emulsions. Pharm Res. 2010; 27:2753-2765), a suitable fluorosurfactant is a block copolymer comprising a perfluorocarbon (perfluoroether) segment and a polyoxyethylene (polyethyleneglycol or PEG) segment, for example and without limitation the triblock copolymer condensation product of a carboxylated perfluoroether with a PEG diamine, such as a Krytox 157FS (FSH or FSL) and PEG block copolymer composition, generally described in Fabilli M L et al.

Poloxamers are nonionic surfactants comprising a polyoxyethylene-polyoxypropylene block copolymer, and include compositions under the trade-name “Pluronic.” Depending on the overall Mw of the block copolymer, and its polyoxyethylene:polyoxypropylene ratio, poloxamers have varying properties (see, e.g., Khaliq N U et al. Pluronic F-68 and F-127 Based Nanomedicines for Advancing Combination Cancer Therapy. Pharmaceutics. 2023 Aug. 9; 15(8):2102), depending on their composition. Pluronic F-68 is used in the exemplary compositions described below, but other poloxamers and/or surfactants, such as nonionic surfactants, may be substituted therefor based on their overall properties, safety, cost, etc. and can be readily formulated and tested for example as described herein.

Lecithins (lecithin compositions) are compositions that occur in plants and animal, but may be natural or synthetic, for example obtained from natural or genetically-modified crops. Non-limiting examples lecithin compositions, such as soy, sunflower, or egg lecithin, include compositions comprising mixtures of glycerophospholipids, such as mixtures of phosphatidylcholine, phosphatidylethanolamine, phosphatidylinositol, phosphatidylserine, and phosphatidic acid.

Lipids include hydrophobic or amphiphilic compounds comprising fatty acid moieties that, as part of an emulsion or emulsifier composition, can help achieve a balance between hydrophobic and hydrophilic compounds to increase the stability of the emulsion. Lipids (e.g., oils) may be a monoglyceride, a diglyceride, and/or a triglyceride (e.g., oils, such as vegetable oils), and may be modified, e.g. esterified, with a hydrophilic moiety, such as a polyoxyethylene (PEG)-containing moiety (for example, a transesterified ethoxylated vegetable oil such as capryl caproyl polyoxyl-8 glycerides (e.g., a LABRASOL©, Gattefosse), and/or oleoyl polyoxyl-6 glycerides (e.g., a LABRAFIL©, Gattefosse)). Modification with a PEG-containing group may be referred to as PEGylation, and a compound modified as such is said to be PEGylated, e.g., a PEGylated oil or a PEGylated vegetable oil.

An effective amount of the emulsifier composition in terms of the emulsions described herein may be a minimum amount of the emulsifier able to produce a stable emulsion. In aspects, an effective amount of the emulsifier is an amount within a range including at its lower end that minimum amount of the emulsifier composition able to produce a stable emulsion and at its upper end an amount of the emulsifier that retains the ability of the emulsion to deliver an effective amount of a gadolinium contrast agent composition to bladder tissue of a patient. The aqueous phase may be present in the emulsion in an amount effective for delivery of a gadolinium contrast agent, and may range from 0.005% to 10% (v/v), and may range from 0.01% to 5% v/v, for example, from 0.1% to 2.5% v/v.

Table A provides an exemplary formulation for the composition described herein. The emulsion without the contrast agent, e.g., gadolinium chelate, may be employed in the bladder as a negative contrast agent for MRI imaging.

TABLE A

| Ingredient | Amount | |
|---|---|---|
| gadolinium chelate | 5-20 | mg/mL |
| perfluorocarbon | 10-20% | w/v |
| emulsifier | 1-2% | w/v |
| surfactant | 1-2% | w/v |
| other lipids (e.g., capryl | 1.5-2% | w/v |

| | |
|---|---|
| caproyl polyoxyl-8 | |
| glycerides and oleoyl | |
| polyoxyl-6 glycerides) | |
| Aqueous phase | Made up to required |
| | volume |

Based on the preceding, a person of ordinary skill in the art can readily determine empirically for any combination of perfluorocarbon, aqueous solution, gadolinium contrast agent, and emulsifier composition (including surfactants and lipidic compounds or compositions) effective to deliver a gadolinium contrast agent to the bladder tissue of a patient from the bladder lumen.

### Examples

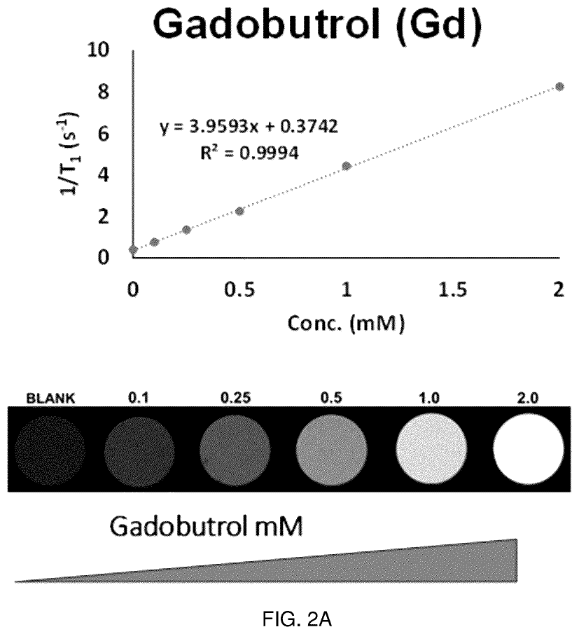

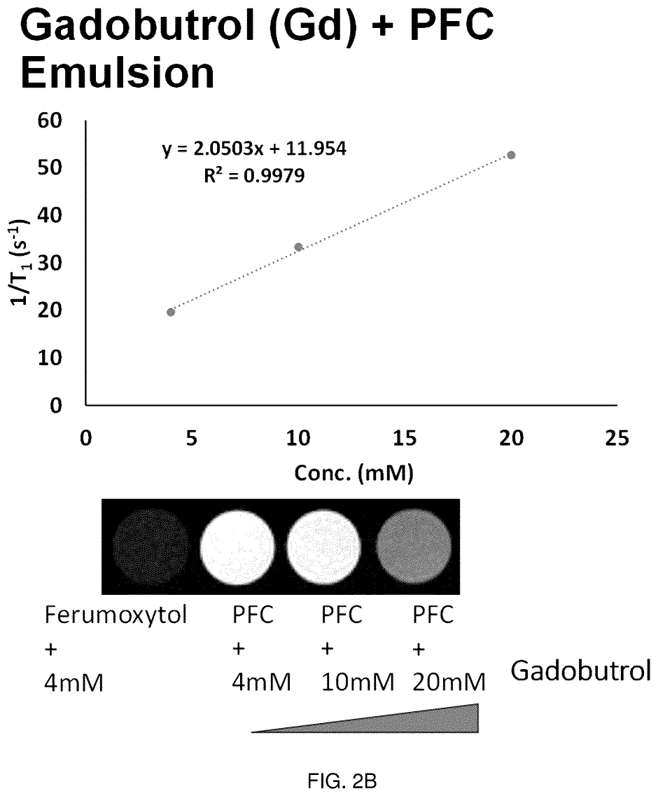

In-vitro and in vivo experiments were conducted, as follows, which, as proof of concept, demonstrate the following:
1) Enhancement of the bladder wall signal in magnetic resonance imaging (MRI) by delivering the contrast agent gadolinium based chelate (Gadobutrol) locally in the bladder as opposed to standard intravenous injection of gadolinium based chelates;
2) The signal enhancement in MRI, clinical standard of water protons (¹H) imaging depends on field strength (0.064 T-11.7 Tesla), and on the relative ratio of the longitudinal (T1) and transverse (T2) relaxation times, both of which are shortened by gadolinium chelates in a concentration dependent fashion (FIGS. 2A and 2B);
3) Since direct bladder instillation of gadolinium based chelate (Gadobutrol) does not confer any advantage over intravenous injection of gadolinium chelates, there has been several attempts made to combine positive contrast from gadolinium chelates with negative contrast of other agents such as air insufflation or iron-oxide particles (Ferumoxytol);
4) the innovation described herein achieves negative contrast in the bladder lumen by mixing high concentration of gadolinium chelates with a ≥10% PFC (perfluorocarbon) emulsion instead of air or iron-oxide particles (Ferumoxytol);
5) The diamagnetic nature of perfluorocarbon (PFC) does not generate artifacts in image field of MRI that are associated with air (a mixture of 78% nitrogen, diamagnetic and 21% oxygen, paramagnetic), blooming artifacts generated by iron, or susceptibility-induced dipole pattern artifacts associated with high concentration of gadolinium chelates or superparamagnetic Ferumoxytol.

It was theorized that PFC instillation in the bladder can obscure the high signal of urine which hinders the true delineation of bladder wall thickness in T2 weighted MRI as perform better than air insufflation or Ferumoxytol instillation in precluding the brightening of the bladder lumen from gadolinium chelate excreted into urine after intravenous injection (see, e.g., Bartolozzi C, et al. MR imaging with STIR technique and air insufflation for local staging of bladder neoplasms. Acta Radiol. 1992 November; 33(6):577-81; PMID: 1449884). Whereas superparamagnetic Ferumoxytol is notorious for causing susceptibility artifacts, magnetic susceptibility of Perfluorodecalin is comparable to that of the bladder wall.

In preliminary work, T1-weighted (gradient echo repetition time TR/echo time 5.23/1.9 ms) and T2-weighted (spin echo TR/TE of 1200/91 ms) images were acquired of bladder phantom constructed of 12% polyacrylamide gel with pore size comparable to umbrella cell tight junctions to mimic the permeability of bladder tissue. To mix gadolinium chelate (Gadobutrol) with oily Perfluorodecalin, a 10% w/v emulsion was prepared essentially as described in Table A before adding ascending concentrations of Gadobutrol, 2-40 mM to 5 mL of emulsion and compared to the negative contrast achieved with 5 mL of Gadobutrol, 2-40 mM mixed with Ferumoxytol (0.1 mM) in bladder phantom.

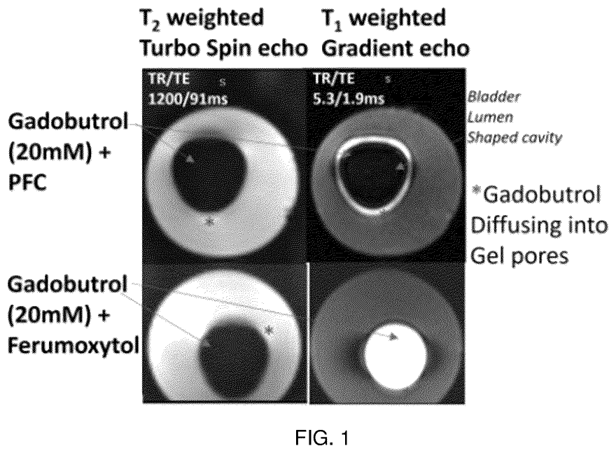

Stokesian diffusion of Gadobutrol is evident from bright circle around cavity darkened by the retention of Ferumoxytol or 4-times larger Perfluorodecalin (FIG. 1). While cavity is only darkened in T2 weighted MRI with Ferumoxytol, Perfluorodecalin darkened cavity in both T1 and T2 weighted images, similar to the use of PFC to fill endorectal coils for generating negative contrast in rectum for the diagnosis of prostate diseases (see, e.g., Choi H, et al. AJR Am J Roentgenol. 2008 April; 190(4):1055-9).

Simultaneous signal enhancement in the bladder wall and signal decay in the bladder lumen are achieved by instilling gadolinium chelate (Gadobutrol) mixed with ≥10% PFC emulsion, to significantly lower T₁ and T₂ water relaxation rate constant (R₁=1/T₁ or R₂=1/T₂, respectively) normalized to concentration of the contrast agent such as Gadobutrol (FIGS. 2A and 2B). While gadolinium chelates are characterized by longitudinal and transverse relaxivity, denoted r₁ and r₂, respectively, (sensitivity to improve image contrast) gadolinium chelates are generally referred to as T₁ agents owing to their larger effect on tissue T₁ than on T₂ for positive image contrast in T₁-weighted images.

To make Perfluorodecalin emulsion, Pluronic F-68 was weighed and added to 100 mL of pure water followed by stirring, using magnetic stirrer at 70° C., 1200 RPM for 15 min. Soya lecithin was weighed, gradually added to the Pluronic F-68 mixture under stirring, followed by addition of capryl caproyl polyoxyl-8 glycerides and oleoyl polyoxyl-6 glycerides one by one. The mixture was stirred at 70° C., 1200 RPM for 15 min followed by gradual addition of perfluorodecalin while stirring (500 RPM) at room temperature, homogenized at 3000-5000 rpm for 5 min. The emulsion was filtered through the Whatman filter paper.

In vitro experiments shown in FIG. 2B demonstrate that ≥10% w/v PFC emulsion alters the relaxation behavior of Gadobutrol 20 mM localized in a tube, proxy for a body cavity like bladder lumen and the localized signal decay (leftmost image in FIG. 2B) approximates the signal decay of Gadobutrol induced by Ferumoxytol 0.1 mM FIG. 1 without magnetic inhomogeneity (MRI artifacts) induced by retention of Ferumoxytol in the bladder lumen.

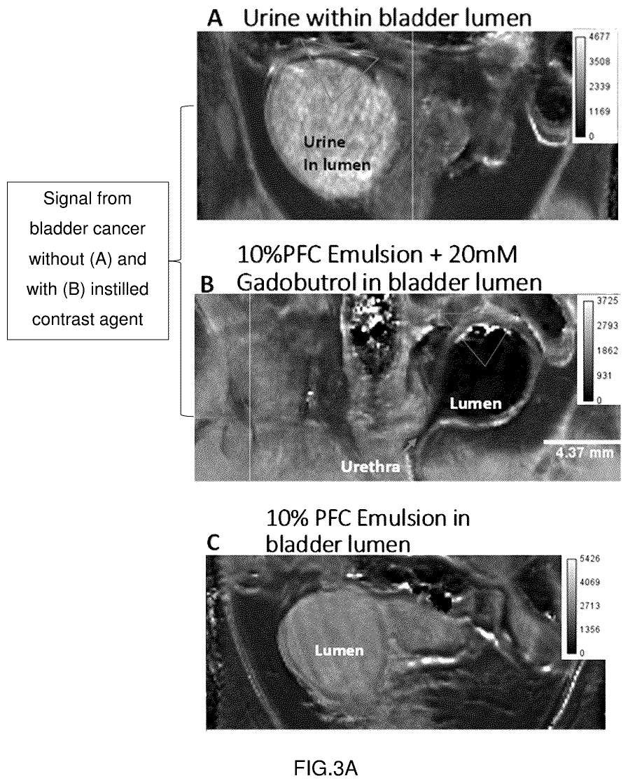

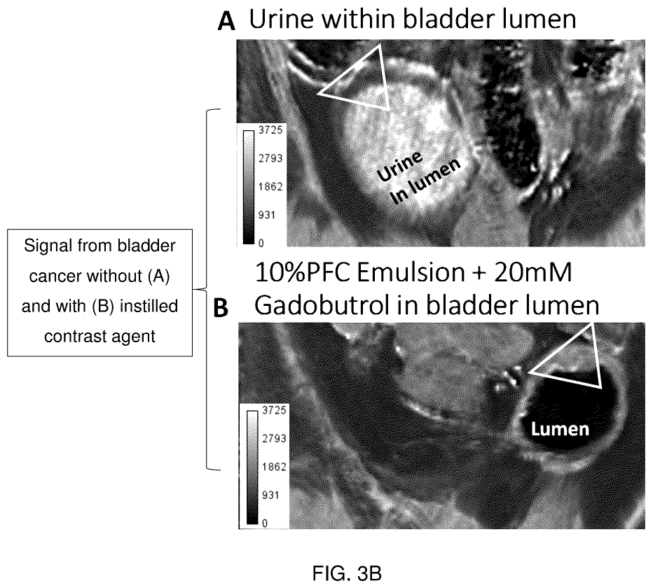

Direct administration of contrast agents in bladder or body cavities: When the data shown in FIG. 2B is viewed in the light of established principles of Stokesian and Fickian diffusion, transurethral administration of Gadobutrol at high concentration of 20 mM in mammalian bladder of live rodents (FIG. 3) confers many advantages: the principle of Stokesian diffusion dictates exclusive paracellular diffusion of Gadobutrol with Stokes-Einstein radius of 0.4 nm across the tight junctions of umbrella cells into inner lining of bladder facing urine to cause signal enhancement while the paracellular diffusion of colloidal particles composing ≥10% w/v PFC emulsion with median Stokes-Einstein radius of >100 nm is retarded to simultaneously cause signal decay within the bladder lumen (right-most tube FIG. 2B).

The signal decay of Gadobutrol 20 mM mixed with ≥10% w/v PFC emulsion shown in FIG. 2B was recapitulated in vivo with mouse model of bladder cancer (FIG. 3A (B)) by dark lumen increasing the conspicuity of bladder tumor growing on inner lining of bladder wall in T1-weighted MRI of mouse bladder immediately after 0.05 mL transurethral instillation of Gadobutrol 20 mM mixed with ≥10% w/v PFC emulsion. Importantly, (FIG. 3A (A)) embodies displays the inadequacy of T1-weighted MRI (without the injection or instillation of contrast agent) to image bladder cancer when urine is present in bladder lumen. See also FIG. 3B.

Simply stated, transurethral instillation of higher concentration of Gadobutrol 10 mM mixed with ≥10% w/v PFC emulsion permits distinct visualization of bladder lesions, such as bladder cancer, for assisting clinical decision making, track the effect of treatment and cancer surveillance. High luminal concentration of Gadobutrol allows the acceleration of Gadobutrol diffusion in accordance with the principle of Fickian diffusion and the steep concentration gradient drives Gadobutrol diffusion into bladder wall to lower millimolar intravoxel concentration, free to exert its unfettered higher relaxivity (FIG. 2A) away from ≥10% w/v PFC emulsion to brighten the tumor or lesion on bladder wall whereas lower relaxivity (FIG. 2B) of entrapped Gadobutrol 20 mM retained in lumen induces local magnetic field inhomogeneities (owing to large magnetic moment of gadolinium chelate) to simultaneously cause signal decay in bladder lumen (FIG. 3A (B)). In this experiment, singular instillation of just gadolinium chelates or ≥10% w/v PFC emulsion (FIG. 3A (C)) did not offer any advantage in T1 weighted MRI.

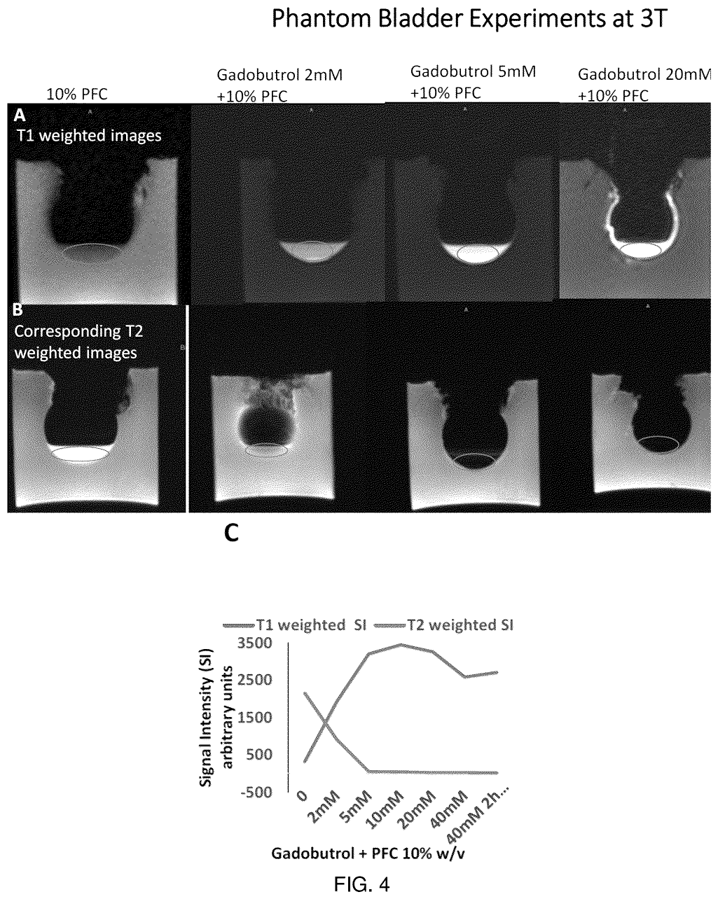

Translation of Clinical field strength: While signal enhancement in MRI (standard ¹H imaging) is dependent on field strength (3 Tesla or 7 Tesla, 3 T and 7 T, respectively), rodent bladder filled to volume of 0.05-0.5 mL can only be successfully imaged at high field strength of 7 T not at 3 T. Therefore, volume of Gadobutrol 10 mM mixed with ≥10% w/v PFC emulsion need to be raised at least 10-fold for predicting imaging performance at more prevalent clinical field strength of 3 T. Accordingly, a proxy of bladder (phantom) constructed with polyacrylamide gel in a bladder shaped spherical cavity filled with 5 mL of ≥10% w/v PFC emulsion was mixed with ascending concentrations of Gadobutrol for T1-weighted (gradient echo) and T2-weighted (spin echo) image acquisition in FIG. 4 (A and B, respectively), respectively. The images taken together display that mixture of Gadobutrol ≥20 mM to ≥10% PFC emulsion in filled space represent the situation of darkened lumen and the steep concentration gradient driving the diffusion of Gadobutrol from high concentration (akin to tubes in FIG. 2B) to differentially lower concentration (akin to tubes in FIG. 2A) into tumor/lesion and normal regions of bladder wall. Overall, ≥10% PFC emulsion attenuates T2-weighted signal intensity marked by complete loss of signal beyond 5 mM while there is a simultaneous accentuation of T1-weighted signal intensity till plateau is reached beyond ≥10 mM, as displayed in FIG. 4 (C). The graph of concentration dependent change in signal intensity shown in FIG. 4 (C) agrees with reported studies of PFC enhancing T1-weighted signal at lower concentrations of gadolinium chelates not viable for localized administration and diffusion dependent signal, therefore FIG. 4 (C) will guide the choice of pulse sequences for darkening the bladder cavity at 3 T while simultaneously brightening the lesion on the bladder wall due to diffusion as displayed by an image of bladder phantom.

The expansion of bright area around filled space containing 5 mL of ≥10% w/v PFC emulsion containing 20 mM of Gadobutrol in T1-weighted image acquired at 2 h (FIG. 1) exemplify the differences in the relaxivity of free Gadobutrol (FIG. 2A) and Gadobutrol entrapped in ≥10% w/v PFC emulsion (FIG. 2B). The expansion of bright area in T1-weighted images as well as the expansion of dark area around filled space in T2-weighted images acquired 2 h later concur with graph of (FIG. 4 (C)) owing to Stokesian diffusion of Gadobutrol and luminal retention of PFC. The principle of Stokesian diffusion dictates the exclusive paracellular diffusion of Gadobutrol with Stokes-Einstein radius of 0.4 nm across the tight junctions of umbrella cells on the inner lining of the bladder facing urine, while the paracellular diffusion of ≥10% w/v PFC emulsion containing particles with median Stokes-Einstein radius of >100 nm is retarded leading to their retention in lumen which we leveraged to brighten lesion and darken lumen, respectively.

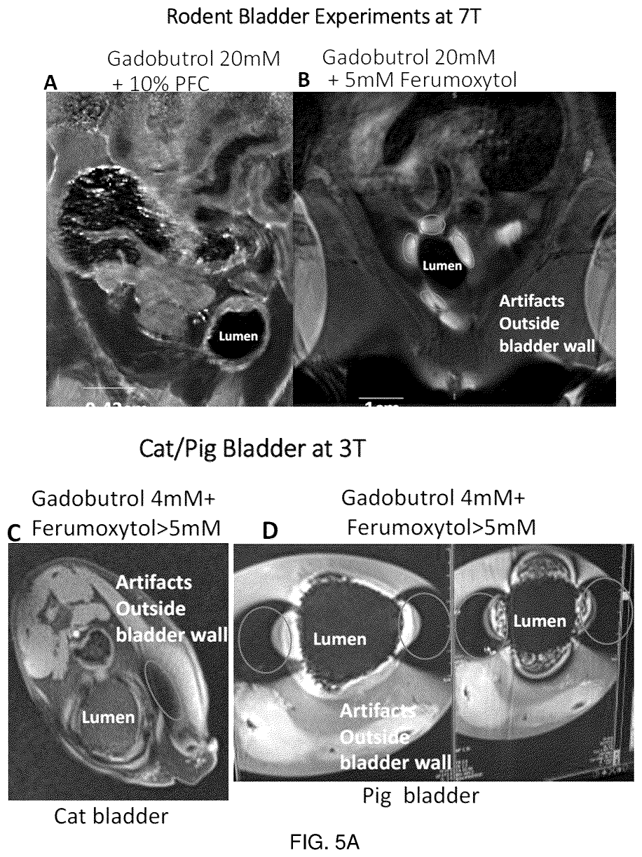

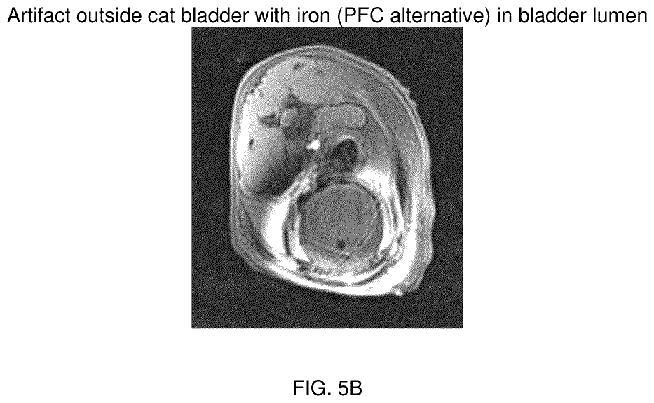

Competitive advantage of PFC emulsion over other agents for negative contrast: A mixture of ≥10% w/v PFC emulsion with 20 mM Gadobutrol generates dark lumen together with bright bladder wall (FIG. 5A (A)) without the susceptibility-induced dipole pattern artifacts outside of the bladder wall associated with the combined instillation of 20 mM Gadobutrol with Ferumoxytol (FIG. 5A (B-D)). The artifacts associated with Ferumoxytol are well known in public domain as described in FDA approved product inserts of similar iron containing products for treating anemia, accordingly, treated patients are advised to avoid MRI for at least 2 weeks after last dose of Ferumoxytol. Indeed, irrespective of the field strength, Ferumoxytol induces MRI artifacts in the image field outside of the bladder wall with spin echo pulse sequence for T2-weighted MRI at 7 Tesla (FIG. 5A (B)) as well as T1-weighted image acquired by gradient echo (single pulse) at 3 Tesla (FIG. 5A (C, D)). In summary, while diamagnetic nature of PFC attenuates (FIG. 5A (A)), superparamagnetic nature of Ferumoxytol accentuates magnetic field inhomogeneity (artifacts) (FIG. 5A (B-D)) which get more pronounced with gradient echo sequences, generally preferred for faster image acquisition in clinically used scanners. See also FIG. 5B.

The present invention has been described in accordance with several examples, which are intended to be illustrative in all aspects rather than restrictive. Thus, the present invention is capable of many variations in detailed implementation, which may be derived from the description contained herein by a person of ordinary skill in the art.

1. A contrast agent emulsion composition comprising at least 10 mM (millimolar) of a contrast agent in an emulsion comprising at least 10% w/v of a perfluorocarbon.

2. The emulsion composition of claim 1, wherein the contrast agent comprises a gadolinium chelate contrast agent.

3. The emulsion composition of claim 2, wherein the contrast agent comprises gadopentetate dimeglumine, gadobenate dimeglumine, gadoxetate disodium, gadofosveset trisodium, gadodiamide, gadoversetamide, gadoterate meglumine, gadoteridol, gadobutrol, or gadopiclenol.

4. The emulsion composition of claim 1, in the form of an emulsion comprising in a final volume: from 5 to 20 mg/ml of a gadolinium chelate, from 10 to 20% w/v of a perfluorocarbon, from 3.5% to 6% w/v of an emulsifier, surfactant, and/or one or more other lipids, and an aqueous phase to the final volume.

5. The emulsion composition of claim 4, wherein the emulsifier, surfactant, and/or one or more other lipids are selected from lecithin, a poloxamer nonionic detergent, and amphiphilic lipid composition comprising a monoglyceride, a diglyceride, and/or a triglyceride modified with a hydrophilic moiety, such as a polyoxyethylene (PEG)-containing moiety, e.g., a transesterified ethoxylated vegetable oil.

6. The emulsion composition of claim 5, comprising: a nonionic surfactant such as a poloxamer nonionic surfactant, and one or both of capryl caproyl polyoxyl-8 glycerides and oleoyl polyoxyl-6 glycerides.

7. The emulsion composition of claim 1, comprising: from 5 to 20 mg/mL of the gadolinium chelate; from 10 to 20% w/v of the perfluorocarbon; from 1 to 2% w/v of lecithin; from 1 to 2% w/v of the poloxamer nonionic surfactant, from 1.5 to 2% w/v of capryl caproyl polyoxyl-8 glycerides and/or oleoyl polyoxyl-6 glycerides; and an aqueous phase, to the final volume.

8. The emulsion composition of claim 1, comprising perfluorodecalin, e.g., ≥10% w/v perfluorodecalin.

9. The emulsion composition of claim 1, comprising perfluorodecalin, lecithin, a poloxamer, and a transesterified ethoxylated oil.

10. A method of imaging an organ having a cavity of a patient, comprising administering an emulsion composition of claim 1 into a cavity of the patient's organ having a cavity, and obtaining a magnetic resonance imaging image of the patient's organ having a cavity containing the emulsion composition.

11. The method of claim 10, wherein the organ having a cavity is a lumen of a patient's bladder.

12. The method of 10, wherein the organ having a cavity is the patient's GI tract, the patient's respiratory tract, the patient's heart, a blood vessel of the patient, or a bursa of the patient.

13. The method of claim 10, wherein the MRI image is obtained using an MRI scanner of at least 1.5 T (1.5 Tesla), or at least 3 T, such as a 7 T MRI scanner.

14. The method of claim 10, wherein the contrast agent comprises a gadolinium chelate contrast agent.

15. The method of claim 14, wherein the contrast agent comprises one or more of gadopentetate dimeglumine, gadobenate dimeglumine, gadoxetate disodium, gadofosveset trisodium, gadodiamide, gadoversetamide, gadoterate meglumine, gadoteridol, gadobutrol, or gadopiclenol.

16. The method of claim 10, wherein the emulsion composition comprises in a final volume: from 5 to 20 mg/ml of a gadolinium chelate, from 10 to 20% w/v of a perfluorocarbon, from 3.5% to 6% w/v of an emulsifier, surfactant, and/or one or more other lipids, and an aqueous phase to the final volume.

17. The method of claim 16, wherein the emulsifier, surfactant, and/or one or more other lipids are selected from lecithin, a poloxamer nonionic detergent, and amphiphilic lipid composition comprising a monoglyceride, a diglyceride, and/or a triglyceride modified with a hydrophilic moiety, such as a polyoxyethylene (PEG)-containing moiety, e.g., a transesterified ethoxylated vegetable oil.

18. The method of claim 16, wherein the emulsion composition comprises: from 5 to 20 mg/mL of the gadolinium chelate; from 10 to 20% w/v of the perfluorocarbon; from 1 to 2% w/v of lecithin; from 1 to 2% w/v of the poloxamer nonionic surfactant, from 1.5 to 2% w/v of capryl caproyl polyoxyl-8 glycerides and/or oleoyl polyoxyl-6 glycerides; and an aqueous phase, to the final volume.

19. The method of claim 10, wherein the emulsion composition comprises perfluorodecalin, such as ≥10% w/v perfluorodecalin.

20. A method of imaging a bladder of a patient, comprising: administering an emulsion composition comprising at least 10% w/v of a perfluorocarbon into the patient's bladder, and obtaining a magnetic resonance imaging image of the patient's bladder containing the emulsion composition.
